(19) United States
(12) Patent Application Publication
(10) Pub. No.: US 2007/0036827 A1
(43) Pub. Date: Feb. 15, 2007

(54) West nile virus vaccine

(75) Inventors: Alexander A. Khromykh, Queensland (AU); Roy Hall, Queensland (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: The University of Queensland, St. Lucia (AU)

(21) Appl. No.: 10/577,866

(22) PCT Filed: Oct. 29, 2004
(86) PCT No.: PCT/AU04/01505

§ 371 Date: Oct. 3, 2006

Related U.S. Application Data
(60) Provisional application No. 60/515,267, filed on Oct. 29, 2003.

(51) Int. Cl. A61K 39/12; A61K 39/193; C12Q 1/70

(52) U.S. Cl. 424/218.1; 977/802; 435/5

(57) ABSTRACT

A vaccine composition and method of administering same is provided that utilizes an isolated nucleic acid corresponding to substantially an entire Kunjin virus which upon administration to an animal is capable of eliciting a protective immune response to another, more pathogenic flavivirus such as West Nile virus NY 99 strain. The isolated nucleic acid may further encode at least one attenuating mutation in a Kunjin virus structural and/or non-structural protein. The isolated nucleic acid may be in the form of DNA linked to a promoter in a plasmid construct for expression in vivo or may be in the form of naked RNA or RNA packaged into VLPs. The composition and method may provide protective immunization of animals such as humans, equines and avians.

### FIELD OF THE INVENTION

THIS INVENTION relates to a composition for prophylactic and/or therapeutic treatment of a flaviviral disease. More particularly, this invention relates to a vaccine for protective immunization against West Nile virus.

### BACKGROUND OF THE INVENTION

West Nile (WN) virus is a mosquito-transmitted flavivirus that produces a potentially fatal disease in humans and horses and has traditionally been associated with viral outbreaks in Europe and Africa (1). In 1999 WN virus appeared for the first time in the New World associated with an outbreak of a fatal or debilitating disease in humans and equines and extremely high levels of morbidity and mortality in several species of native birds in New York (2). Since its introduction into North America, WN virus has spread to most states of the USA and to parts of Canada and Mexico via mosquito-bird transmission cycles (3; www.cdc.gov/ncidod/dvbid/westnile/index.htm). There is now an urgent need for an effective vaccine for veterinary and medical prophylaxis.

Kunjin (KUN) virus is a genetically stable Australian flavivirus originally shown to be very closely related antigenically (by virus neutralization tests and monoclonal antibody binding studies) to the Sarafend strain of WN virus (4, 5) and genetically to the prototype Ugandan strain (6, 7). When the North American WN virus strain New York 99 (NY99) was isolated, comparative studies (2, 7) revealed 98 to 99% amino acid homology with KUN virus throughout the coding sequence, compared to 93% homology for the Ugandan strain. Because of the homology with WN NY99 and other lineage 1 strains of WN virus (8), KUN virus was re-classified as a subtype of WN virus by the International Committee for the Taxonomy of Viruses (9). However, unlike WN virus, KUN produces only rare non-fatal cases of human and equine disease (10). Indeed, comparative studies of WN virus strain NY99 and KUN virus have revealed that 1000-10,000 fold more infectious virus of the latter is required to produce disease in adult mice by peripheral inoculation (11).

### SUMMARY OF THE INVENTION

The invention is broadly directed to use of an infectious flavivirus to protectively immunize against at least one other flavivirus.

Preferably, a less pathogenic flavivirus is used to immunize against at least one other more pathogenic flavivirus.

In a first aspect, the invention provides an immunotherapeutic composition comprising an isolated nucleic acid capable of producing an infectious Kunjin virus, which upon administration to an animal, elicits a protective immune response to at least another flavivirus.

Preferably, said isolated nucleic acid encodes at least one attenuating mutation in a Kunjin virus non-structural protein encoded thereby.

In a second aspect, the invention provides a method of immunization including the step of administering an isolated nucleic acid capable of producing an infectious Kunjin virus to an animal to thereby elicit a protective immune response to at least another flavivirus.

In a third aspect, the invention provides a method of using Kunjin virus to identify another flavivirus against which Kunjin virus is suitable for use as an immunogen, said method including the steps of:

(i) administering an isolated nucleic acid capable of producing an infectious Kunjin virus to an animal; and

(ii) determining whether said animal is protectively immunized against infection by another flavivirus;

wherein if said animal is protectively immunized against said another flavivirus, Kunjin virus is suitable for use as an immunogen against said another flavivirus.

Preferably, said another flavivirus is more pathogenic than Kunjin virus.

In a fourth aspect, the invention provides a non-human animal immunized according to the method of the second aspect.

In a fifth aspect, the invention provides an immunocompetent biological material isolated from an animal immunized according to the method of the third aspect.

In particular embodiments, the immunocompetent biological material may be an immune cell or antigen-presenting cell, an antibody, blood, plasma or serum isolated from the animal.

In a preferred embodiment of the aforementioned aspects, said at least another flavivirus is a strain of West Nile virus other than Kunjin virus.

In a particularly preferred embodiment, said strain of West Nile virus is NY99 strain.

Throughout this specification, unless otherwise indicated, “comprise”, “comprises” and “comprising” are used inclusively rather than exclusively, so that a stated integer or group of integers may include one or more other non-stated integers or groups of integers.

### BRIEF DESCRIPTION OF THE FIGURES

FIG. 1. Replication kinetics of Kunjin virus mutants in C6/36 cells (immortalized mosquito cell line). The Kunjin viruses analyzed in this study are: 250PRO (wild-type virus clone), FLSD (leucine at residue 250 of NS1), 250A (alanine at residue 250 of NS1), 250V (valine at residue 250 of NS1).

FIG. 2. Replication kinetics of Kunjin virus mutants in Vero cells (immortalized african green monkey kidney cell line). The Kunjin viruses analyzed in this study are: 250PRO (wild-type virus clone), FLSD (leucine at residue 250 of NS1), 250A (alanine at residue 250 of NS1), 250V (valine at residue 250 of NS1).

FIG. 3. Vero cells were infected with KUN wt virus (WT), and KUN virus with Ala30 to Pro-mutated NS2A gene (NS2A/A30P) at MOI=0.01-4d, 6d and 8d after infection, cells were fixed and stained with crystal violet (A). To quantify cytopathic effect (CPE), stained cells were lysed with 100% methanol and the released crystal violet was measured at OD₆₂₀. The percentage of CPE was calculated according to the formula: 100%-[(OD₆₂₀infected sample/OD₆₂₀uninfected samples)×100%].

FIG. 4. Induction of IFN-β specific mRNA by KUN virus in A549 cells. Northern blot analysis of IFN-β mRNA was performed with total cellular RNA isolated from A549 cells infected by KUN viruses. Normal A549 cells and A549 cells were infected with KUN virus encoding either wild type NS2A gene (WT) or Ala30 to Pro-mutated NS2A gene (NS2A/A30P) at MOI=1 for 24 hours. Total cellular RNA were prepared and ˜10 μg of RNA was used for northern blot hybridization with radiolabeled probes specific for IFN-β, KUN replicon RNA and β-actin mRNAs.

### DETAILED DESCRIPTION OF THE INVENTION

The present invention, at least in part, arises from the unexpected finding that immunization of mice with a plasmid DNA encoding full-length Kunjin virus (KUN) cDNA under the control of the cytomegalovirus (CMV) promoter, confers protective immunity against NY99 strain of West Nile virus.

Furthermore, inclusion in pKUN1 DNA of a mutation in the KUN NS1 gene ensured significant attenuation of pKUN1-derived virus while inducing protective immunity against KUN virus and the NY99 strain of West Nile virus without causing disease.

The present invention provides the first ever report of protective immunization by administration of a DNA copy of a flavivirus genome, and more particularly, immunization by a non-pathogenic flavivirus genome (Kunjin) against a far more virulent and pathogenic flavivirus (NY99 strain of West Nile virus).

Use of plasmid DNA encoding a functional viral genome as a vaccine eliminates a number of concerns associated with preparations of live viral vaccines, including possible contaminations with endogenous viruses during preparation of vaccine in cell cultures, and the instability of enveloped viruses at ambient temperatures.

The invention in one aspect therefore provides use of an isolated nucleic acid that is capable of producing an immunogenic, infectious Kunjin virus for immunization against at least one other flavivirus, preferably a more pathogenic flavivirus.

In particular embodiments, the invention also provides use of a Kunjin virus nucleic acid that has one or more attenuating mutations that confer reduced cytopathicity and hence, increased safety, without compromising immunity induced by the Kunjin virus nucleic acid.

It is also noted that, at least in so far as an NS2A Ala30 to Pro mutation is concerned, induction of β-interferon mRNA is enhanced relative to wild-type, which induction may attenuate virus replication in vivo and enhance anti-viral responses.

The term “nucleic acid” as used herein designates single- or double-stranded mRNA, RNA, and DNA inclusive of cDNA and genomic DNA.

By “protein” is meant an amino acid polymer. Amino acids may include natural (i.e genetically encoded), non-natural, D- and L-amino acids as are well known in the art.

A “peptide” is a protein having less than fifty (50) amino acids.

A “polypeptide” is a protein having fifty (50) or more amino acids.

As used herein, “flavivirus” and “flaviviral” refer to members of the family Flaviviridae within the genus Flavivirus, which contains 65 or more related viral species. Typically, flavivirus are small, enveloped RNA viruses (diameter about 45 nm) with peplomers comprising a single glycoprotein E. Other structural proteins are designated C (core) and M (membrane-like). The single stranded RNA is infectious and typically has a molecular weight of about 4×10⁶ with an m7G ‘cap’ at the 5′ end but no poly(A) tract at the 3′ end; it functions as the sole messenger. Flaviviruses infect a wide range of vertebrates, and many are transmitted by arthropods such as ticks and mosquitoes, although a separate group of flaviviruses is designated as having no-known-vector (NKV).

Particular, non-limiting examples of flavivirus are West Nile virus, Kunjin virus, Yellow Fever virus, Japanese Encephalitis virus, Dengue virus, Montana Myotis leukoencephalitis virus, Usutu virus, St Louis Encephalitis virus and Alkhurma virus.

The West Nile virus subgroup somewhat controversially includes Kunjin virus as a sub-type. For convenience and without necessarily agreeing with this classification, according to this specification Kunjin virus is an example of a first West Nile virus strain.

It will be appreciated that the present invention contemplates use of an isolated nucleic acid capable of producing an infectious Kunjin virus to protectively immunize against at least one other, more virulent and/or pathogenic flavivirus.

The invention may therefore be useful in human and veterinary medicine, given that West Nile virus is capable of causing disease in humans and non-human animals such as equines and avians.

A preferred pathogenic flavivirus is any West Nile virus strain that is capable of causing fatal disease in mammals.

In one particular embodiment, the mammal is a human.

In another particular embodiment, the mammal is a non-human mammal such as an equine.

As used herein, “equine” refers to any member of the genus Equus, which includes and encompasses Equus burchelli, the plains zebra of Africa, Equus zebra, the Mountain zebra of South Africa, Equus grevyi, Grevy's zebra, Equus caballus, the true horse; Equus hemionus: the desert-adapted onagers of Asia & the Mideast; and Equus asinus the true asses & donkeys of northern Africa.

NY99 strain is a preferred example of a second West Nile virus strain.

Therefore, in a preferred embodiment the invention provides a vaccine comprising an isolated nucleic acid corresponding to substantially an entire genome of Kunjin virus, which upon administration to an animal, elicits a protective immune response to at least NY99 strain of West Nile virus.

In light of the foregoing, it will be appreciated that the present invention may be applicable to using Kunjin virus to immunize against flaviviruses other than West Nile virus.

For example, Kunjin virus could be used to immunize against Japanese Encephalitis Virus (JEV), Yellow Fever Virus (YFV) or St Louis Encephalitis Virus (SLFV), although without limitation thereto.

According to one embodiment of the invention, the isolated nucleic acid of the first flavivirus is an RNA molecule corresponding to substantially the entire genome of Kunjin virus.

The RNA molecule may be substantially pure “naked” RNA or may be packaged into virions.

According to another embodiment of the invention, the isolated nucleic acid of the Kunjin virus is a cDNA copy of substantially the entire RNA genome of said Kunjin virus.

In a particularly preferred form, the isolated nucleic acid nucleic acid is a cDNA copy of a Kunjin virus genome.

In light of the foregoing, it will be understood that “substantially the entire genome” encompasses isolated nucleic acids having minor deletions or sequence alterations with respect to the Kunjin virus genome that do not significantly reduce the ability of the isolated nucleic acid to produce infectious virus.

In constructing the preferred Kunjin virus cDNA copy described herein, the inventors used an FLSDX clone, the sequence of which differs from the parental MRM61C strain Kunjin virus sequence originally deposited in Genbank.

The sequence differences, both at the nucleotide and amino acid level, are set forth in Table 4.

The invention therefore provides at least an infectious fragment of an isolated Kunjin virus RNA genome or cDNA copy thereof, having at least one of the nucleotide sequence variations set forth in Table 4.

In particular embodiments, nucleotide 554 is C, nucleotide 2556 is G, nucleotide 2924 is G, nucleotide 3218 is T, nucleotide 5996 is T, nucleotide 6017 is T and nucleotide 7112 is A, numbered with reference to the published MRM61C sequence.

In a preferred form, the invention contemplates the presence of one or more attenuating mutations in the isolated nucleic acid corresponding to at least a substantial portion of the Kunjin virus genome.

By “attenuating mutation” is meant a mutation that decreases, inhibits, suppresses or otherwise reduces the replication competence of the flavivirus and/or its ability to produce disease in vivo.

It will be appreciated that the Kunjin virus nucleic acid used in the vaccine of the invention suitably encodes nonstructural proteins NS1, NS2A, NS2B, NS3, NS4A, NS4B and NS5.

Examples of amino acids that may be attenuated include Proline residue 250 of nonstructural protein NS1, Alanine residue 30 of nonstructural protein NS2A, Asparagine residue 101 of nonstructural protein NS2A and Proline residue 270 of nonstructural protein NS5, although without limitation thereto.

In one particular embodiment, proline residue 250 of nonstructural protein NS1 is substituted by an amino acid selected from the group consisting of leucine, valine and alanine.

In another particular embodiment, Alanine 30 of nonstructural protein NS2A is substituted by Proline.

In yet another particular embodiment, Asparagine 101 of nonstructural protein NS2A is substituted by Aspartate.

In still yet another particular embodiment, Proline 270 of nonstructural protein NS5 is substituted by Serine.

With regard to attenuating mutations in non-structural proteins, Kunjin virus NS2A, NS2B, NS3, NS4A and NS4B are involved in inhibition of IFN-α, -β and -γ signaling (ref 32; data not shown) Thus mutations in any of these proteins that increase virus sensitivity to IFNs should lead to virus attenuation.

It will also be appreciated by persons skilled in the art that other mutations at the abovementioned residues may be used, as well as mutations and/or deletions in other regions, for example untranslated regions, C gene, prM gene, E gene, and other nonstructural genes providing that they enable the production of infectious Kunjin virus with an attenuated phenotype.

A particular Kunjin virus E protein substitution contemplated by the present invention is Glu390 to Gly (ref 31).

Other possible attenuating mutations in E protein are gain-of-net-positive-charge amino acid changes at residues 49, 138, and 306. All of these mutations should increase positive charge and thus improve virus binding to cells (mainly liver cells) leading to quicker clearance of the virus from blood circulation. This should lead to decreased ability of virus to reach the brain and cause encephalitis.

It will also be appreciated that each of the above mutations or substitutions may be present singly or in combination in a Kunjin virus immunogenic nucleic acid of the invention.

According to one form of the present invention, said Kunjin virus nucleic acid is present in a “genetic construct” which includes within its scope an “expression vector” wherein the isolated nucleic acid of said Kunjin virus nucleic acid is operably linked or operably connected to one or more regulatory sequences that initiate, control, terminate or otherwise regulate transcription and/or translation of the Kunjin virus nucleic acid and/or encoded proteins.

Such an expression construct may be a DNA construct comprising a promoter that facilitates transcription of said Kunjin virus nucleic acid in a mammalian cell.

In another embodiment, an expression construct may be a DNA construct comprising a promoter that facilitates transcription of said Kunjin virus nucleic acid in vitro for subsequent delivery as an immunogen.

A preferred promoter for in vitro transcription of RNA from a DNA expression construct is an SP6 promoter.

A preferred promoter for in vivo transcription of RNA from said DNA expression construct in mammalian cells is a cytomegalovirus (CMV) promoter. However, it will be appreciated that other well-known promoters active in mammalian cells are contemplated, including an SV40 promoter, a human elongation factor alpha promoter and an alpha crystallin promoter, although without limitation thereto.

The genetic construct may further comprise the antigenomic sequence of the hepatitis delta virus ribozyme (HDVr) and polyadenylation signal from simian virus 40 (pA) inserted downstream of the 3′UTR to ensure production of KUN RNA molecules with precise 3′termini for efficient initiation of replication.

In a particularly preferred embodiment, the genetic construct is pKUN1.

In an even more preferred embodiment, the genetic construct is pKUN1 encoding one or more attenuating mutations as hereinbefore described.

Immunotherapeutic Compositions, Vaccines and Methods of Immunization

A particular aspect of the invention relates to use of an isolated nucleic acid capable of producing infectious Kunjin virus to protectively immunize an animal against at least one another flavivirus.

Accordingly, an immunotherapeutic composition or vaccine of the invention may be in any of the following forms:

(i) an RNA transcribed from a DNA expression construct;

(ii) a DNA expression construct of the invention directing transcription of replicating RNA in vivo; or

(iii) secreted virions produced by transfecting the above RNA or DNA into a permissive cell line such as BHK-21 or Vero, although without limitation thereto.

Virions as described above may be used as a live or killed vaccine as is well understood in the art.

The immunotherapeutic composition may further comprise an acceptable carrier, diluent or excipient and, optionally, an adjuvant.

By “acceptable carrier, diluent or excipient” is meant an additional substance that is acceptable for use in human and/or veterinary medicine, with particular regard to immunotherapy.

By way of example, an acceptable carrier, diluent or excipient may be a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

Any safe route of administration may be employed for providing a patient with the composition of the invention. For example, oral, rectal, parenteral, sublingual, buccal, intravenous, intra-articular, intramuscular, intra-dermal, subcutaneous, inhalational, intraocular, intraperitoneal, intracerebroventricular, transdermal and the like may be employed. Intra-muscular and subcutaneous injection may be appropriate, for example, for administration of immunotherapeutic compositions, proteinaceous vaccines and nucleic acid vaccines. It is also contemplated that microparticle bombardment may be particularly useful for delivery of nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Immunotherapeutic compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

Immunotherapeutic compositions of the invention may be used to prophylactically or therapeutically immunize animals such as humans.

However, veterinary applications in other animals are contemplated, preferably vertebrate animals including domestic animals such as livestock and companion animals.

Preferably, the vertebrate animal is a mammal.

In one particular embodiment, the mammal is a human.

In another particular embodiment, the mammal is a non-human mammal such as an equine.

In other embodiments, the vertebrate animal is an avian such as a crow, which is a known vector for WN virus.

The protective immune response elicited by the composition or vaccine of the invention may include humoral and/or cellular responses, such as by induction of antibodies, CD8+ CTLs and/or CD4+ T cells.

It will also be appreciated that immunotherapeutic compositions and vaccines of the invention may, in certain embodiments, include an adjuvant.

As will be understood in the art, an “adjuvant” means one or more substances that enhances the immunogenicity and/or efficacy of a vaccine composition. Non-limiting examples of suitable adjuvants include squalane and squalene (or other oils of animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran or with aluminium phosphate; carboxypolymethylene such as Carbopol′ EMA; acrylic copolymer emulsions such as Neocryl A640 (e.g. U.S. Pat. No. 5,047,238); vaccinia or animal poxvirus proteins; sub-viral particle adjuvants such a cholera toxin, or mixtures thereof.

The present inventors propose that immunization with Kunjin virus will produce protective, humoral and/or cell-mediated immunity against other flaviviruses such as West Nile virus.

The present invention therefore contemplates immunocompetent, biological material isolated from an animal immunized according to the invention.

In one embodiment, the immunocompetent, biological material is one or more immune cells or antigen-presenting cells isolated from an animal immunized according to the present invention. Isolated cells may be dendritic cells, macrophages and other antigen-presenting cells, T cells, B cells or any other cells that are involved in the initiation, regulation and/or maintenance of cell mediated immunity.

Isolated immune cells or antigen-presenting cells may have efficacy in dendritic cell therapy, production of monoclonal antibodies and adoptive transfer in passive immunization, for example, although without limitation thereto.

In another embodiment, the immunocompetent, biological material is blood, plasma or serum isolated from an animal immunized according to the present invention.

Isolated blood, plasma or serum may have particular uses in relation to isolation and purification of anti-flavivirus antibodies and in passive immunization, for example, although without limitation thereto.

So that the invention may be readily understood and put into practical effect, the skilled person is directed the following non-limiting examples.

### EXAMPLES

### Example 1

### Kunjin Virus Induces Protective Immunity to NY99 Strain West Nile Virus

### Materials and Methods

Cell Culture and Virus Preparations.

Vero cells were grown in HEPES-buffered Medium 199 (Gibco) supplemented with antibiotics and 10% FBS and incubated at 37° C. C6/36 ceLls were cultured in Medium 199 supplemented with antibiotics and 10% FBS and incubated at 28° C. and 5% C0₂. For virus stock production, Vero cells were infected with KUN virus (MRM61C strain; 4), WN virus (NY99-4132 strain, obtained from the Division of Vector-Borne Infectious Diseases, Centers for Disease Control, Fort Collins ), or FLSD virus (derived from KUN cDNA clone FLSD; 13, 15) at a multiplicity of infection of 0.1-1 and cultured in medium supplemented with 2% FBS. Culture supernatant was harvested and clarified at 72-96 h post-infection when 50-70% of cells showed cytopathic effects (CPE). The concentration of infectious virus in stocks was determined by titration on Vero cells in 96-well plates and calculated as the 50% infectious dose (ID₅₀)/ml (13). One ID₅₀ is equivalent to one infectious unit (i.u.).

Plasmid DNA Constructs.

Construction of plasmid DNAs pKUN1, coding for the infectious full-length KUN RNA, and pKUN1 dGDD, coding for the non-replicating full-length KUN RNA, was described previously (12). In these plasmids, the KUN cDNA sequence is placed under the control of cytomegalovirus early promoter-enhancer region (CMV) to allow in vivo transcription of KUN RNA by the cellular RNA polymerase II. The plasmids also have the hepatitis delta virus ribozyme sequence (HDVr) inserted immediately after the last nucleotide of KUN cDNA sequence to ensure production of KUN RNAs with the precise 3′ terminus, which was shown to be beneficial for more efficient RNA replication (16). The KUN genome in pKUN1 and pKUN1dGDD plasmids is derived from the full-length cDNA clone FSDX, that has recently been fully sequenced (17; GeneBank accession No AY274504). The sequence of FLSDX and FLSD cDNA clones is the same and both contain Pro to Leu substitution at amino acid 250 in the NS1 gene. FLSDX clone has 6 other conservative amino acids substitutions compared to the published sequence of MRM61C strain of KUN virus (17) that did not appear to affect the growth properties of recovered virus.

KUNVNS1250Ala and KUNVNS1250Val viruses were generated by substituting Alanine or Valine respectively for Proline at residue 250 in NS1 in the full-length cDNA clone of KUN virus FLSDX(pro). Mutant virus was produced in BHK cells after transfection with KUNVNS1250Ala or KUNVNS1250Val RNA and titrated by plaque assay on Vero cells to determine the virus titre

Mouse Immunization and Challenge.

Groups of five to 12 BALB/c mice at 4-5 wks of age were immunized with a total of 0.1, 1 or 10 ug of pKUN1 DNA injected intramuscularly (i.m.) in 2 sites of the thigh muscles. Groups of mice were similarly immunized with 1 or 10 ug of defective DNA (pKUN1dGDD). An additional group was immunized by the intra-peritoneal (i.p.) route with a sub lethal dose of 10³ infectious units (i.u.) of attenuated FLSD virus (contains the same genomic sequence as pKUN1 progeny virus) and one group remained unimmunised. At 21 days post immunization, mice from each group were challenged by the intra-cerebral (i.c.) route with 10³ i.u. of the wild type KUN virus (MRM61C strain). Similarly, immunized mice were also challenged with 20 i.u. of West Nile virus (NY99-4132 strain) by either i.c or i.p. route.

The appropriate challenge doses for KUN and WN viruses in 7-8 week old BALB/c mice were previously determined in a pilot study (results not shown). It was observed that i.p. inoculation of 20 i.u. of WN virus was sufficient to produce clear disease symptoms (severely ruffled fur and lethargy) in all animals by 7-8 days post infection. Of these, approximately 60-70% progressed to a more severe form of disease (flaccid hind leg paralysis, hunched posture, moribund ) and were euthanased. In contrast, relatively high doses of the wild type KUN virus inoculated i.p. did not produce consistent rates of morbidity or mortality in these mice. By the i.c. route 20 i.u of WN and 1000 i.u. of KUN viruses were sufficient to produce 80-100% mortality in inoculated mice.

Detection of KUN Virus in Immunized Mice.

For virus detection, 3 mice from each group was tail bled on days 3, 4, 5, 6 and 7 post immunisation under aseptic conditions and blood was allowed to clot overnight at 4° C. The serum was then separated, snap frozen and stored at −70° C. until tested. On the day of analysis sera were rapidly thawed then kept on ice. 25 ul of pooled undiluted sera were inoculated in duplicate onto fresh monolayers of C6/36 cells in 96 well plates. After 7 days inoculation the culture supernatant was then carefully removed and replaced with an equal volume of 20% acetone in PBS and the cells allowed to fix for 2 h at 4° C. The fixative was then removed and plates dried at 37° C. Viral antigens were then detected by ELISA (18) using anti-KUN rabbit antiserum (19). Supernatants from inoculated C6/36 cells were further passaged onto fresh monolayers of C6/36 cells and the process repeated to detect progeny virus.

Measurement of Serum Antibody Responses.

For detection of virus-specific antibody in sera, mouse blood was collected by tail bleed at 19 days post inoculation, held at 4° C. overnight to clot, serum was separated and snap frozen at −70° C. until tested. Sera from each mouse group were pooled and titrated in doubling dilutions in fixed-cell ELISA against the viral proteins of wild type KUN virus and WN virus as previously described (20). The reciprocal of the serum dilution that produced an OD of at least 0.3 on viral antigen and at least 0.2 higher than that produced on control antigen (fixed uninfected cells) was deemed the ELISA titre of each sample.

Microneutralization Assays.

Sera samples were tested for neutralization of KUN and WN viruses by microneutralisation assay as described previously (21). Briefly, sera from each mouse group were pooled, heat-inactivated at 56° C. and serially diluted two-fold in cell growth medium. Twenty-five ul of each dilution was then added in duplicate to wells of a 96 well culture plate. An equal volume of growth medium containing approximately 100 i.u. of virus was then added to each well, and plates were allowed to incubate at 37° C. with occasional gentle agitation. Fifty ul of growth media containing approximately 10⁴ Vero cells were then added to each well and plates incubated at 37° C. for 5 days. The reciprocal of the serum dilution that inhibited the formation of viral cytopathic effects was deemed the neutralization titre.

Kunjin Virus Replication Kinetics

Vero cells (african green monkey kidney cells) and C6/36 (Aedes albopictus) cells cultured in 12-well plates were infected with either wild-type Kunjin virus clone 250PRO (proline at residue 250 of NS1), FLSD (leucine at residue 250 of NS1), 250A (alanine at residue 250 of NS1) or 250V (valine at residue 250 of NS1) at a multiplicity of infection (MOI) of 0.1. Two hours post-infection the culture fluid was removed from the cells and they were washed twice with 2 mL of PBS. 2 mL of fresh media supplemented with 2% FBS was added to each well and samples were harvested at regular intervals until 72 hours post-infection. The samples were stored immediately at −70° C.

Titration of Viruses

Serial ten-fold dilutions were made from the virus sample in culture media and 50 μL of each dilution of virus was added to 10 wells of a 96 well plate containing confluent vero cells. At 7 days post-infection the wells of the plate exhibiting cytopathic effect (CPE) were recorded and this data was used to calculate the 50% tissue culture infectious dose (TCID50) of the initial virus sample using the Reed-Muench equation. Fixed-cell ELISA was used to corroborate the TCID50 data obtained by visual inspection of CPE.

### Results

Replication Kinetics of Virus Mutants at Amino Acid 250 in the NS1 Protein.

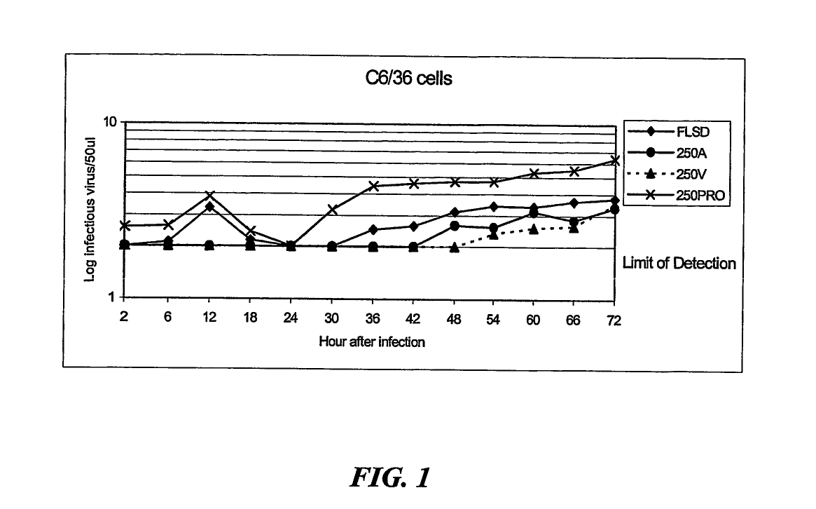

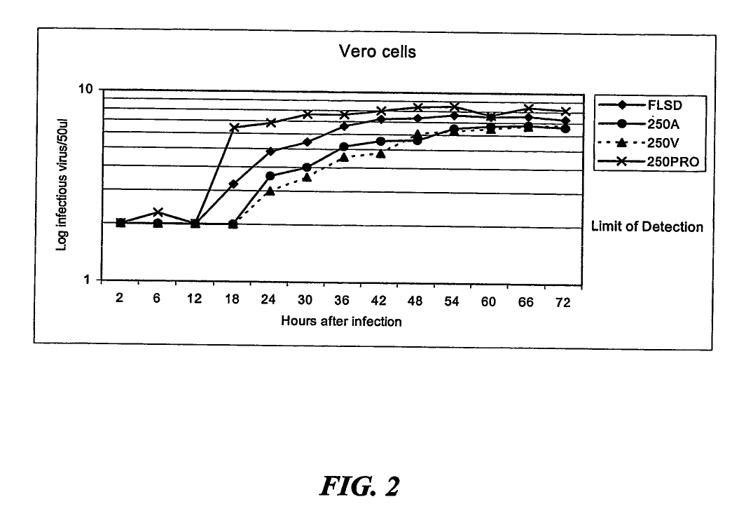

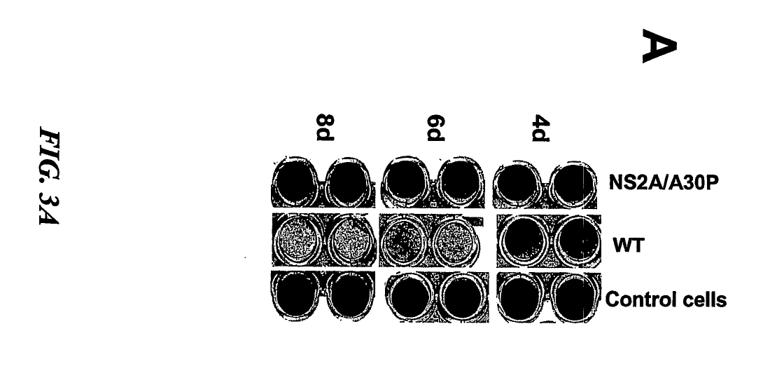

Proline to leucine, alanine or valine mutations at residue 250 of NS 1 were investigated with regard to Kunjin virus replication kinetics in C6/36 cells (FIG. 1) and in Vero cells (FIG. 2). The data show that all three mutations at residue 250 resulted in delayed viral replication, with the valine mutation resulting in the lowest level of infectious virus and slightly longer delay than the alanine mutation. It is proposed that either of these NS1 mutations could be used for attenuating Kunjin virus for the purpose of immunizing against another flavivirus according to the present invention.

Injection of pKUN1 DNA Induces Low Level Viraemia in Mice.

We showed previously that transfection of pKUN1 DNA directing transcription of replication-competent full-length KUN RNA but not of pKUN1dGDD DNA directing transcription of replication-deficient full-length KUN RNA into BHK cells resulted in production of secreted infectious KUN virus (12). We examined whether injection of pKUN1 DNA into mice would lead to production of infectious virus in vivo. Groups of mice were injected i.m. with different doses of pKUN1 DNA or injected i.p. with a sublethal dose (1000 i.u.) of attenuated KUN FLSD virus. FLSD virus was prepared in BHK cells transfected with FLSD RNA (13) that has a genomic sequence identical to that expected to be present in the pKUN1-derived virus and containing proline to leucine mutation at amino acid 250 in the NS1 protein. The sera from injected mice were tested for the presence of infectious KUN virus between 3 and 7 days post injection, by inoculation of C6/36 and Vero cell cultures.

KUN virus was isolated after the second passage of culture supernatant from mosquito cells initially inoculated with undiluted mouse serum collected at 4 days post injection with 0.1 ug of pKUN1 DNA, and at 3 days post injection with 1 ug of pKUN1 DNA (Table 1). In the group injected i.p. with FLSD, progeny virus was detected after secondary passage of mouse serum samples collected at 3, 4, and 5 days after FLSD injection. No virus was isolated from mice that received 10 ug of pKUN1 DNA or 10 ug of pKUN1 dGDD (defective) DNA. Although serum samples were not titrated, the observation that virus was not detected in the first passage on C6/36 cells of undiluted mouse sera collected from each experimental group, indicated that only trace amounts of virus were present in samples positive on the second passage.

Analysis of virus recovered from animals vaccinated i.m. with pKUN1 DNA confirmed that the attenuating leucine mutation at NS1 residue 250 was retained, along with the monomeric NS1 phenotype (results not shown). In addition, the nucleotide sequence spanning the entire NS1 gene (approximately 1000 nucleotides) was identical with the corresponding sequence of the pKUN1 DNA used to immunize the animals, indicating that the viral RNA was transcribed from the plasmid with high fidelity in vivo (results not shown).

The data presented herein demonstrate that i.m. injection of as little as 0.1 ug of pKUN1 DNA resulted in production of infectious, but non-lethal KUN virus in adult mice.

Immunization with pKUN1 DNA Induces Neutralizing Antibody to New York Strain of WN Virus.

Nineteen days after immunization, mice were bled and sera from each group pooled and tested for antibody to wild type KUN virus in ELISA and by micro-neutralization assay. Sera from mice immunized i.p. with 1000 i.u. of KUN FLSD virus showed the highest titer to KUN antigens in ELISA (1280), while sera from mice receiving i.m. injection of 0.1, 1, or 10 ug of pKUN 1 DNA displayed ELISA titers of 160, 320, and 320 respectively (Table 2). KUN-specific antibody responses in unimmunized mice or mice receiving defective DNA could not be detected (ELISA titer<40). Neutralizing antibody to KUN virus was also detected in sera of mice immunized with pKUN1 DNA (titers of 10-20) or FLSD virus (40). Slightly higher levels of antibody as measured by ELISA and virus neutralization correlated with a longer period of viraemia in FLSD-immunized mice (see Table 1).

Sera from mice immunized with FLSD virus and with KUN plasmid DNAs were then tested for reactivity with viral antigens of the New York strain of WN virus in ELISA and for neutralization of the New York strain of WN virus in vitro. Slightly lower ELISA titers to WN antigen compared to that of KUN antigen were recorded for mice immunized with each dose of pKUN1 DNA (80) or FLSD virus (640). However similar neutralizing titers were observed (Table 2).

Thus, the results presented in this section clearly demonstrate that immunization with pKUN1 plasmid DNA induces cross-reactive antibodies that are able to neutralize the New York strain of WN virus with efficiency similar to that observed for neutralization of KUN virus.

Immunization with pKUN1 DNA Protects Mice Against the New York Strain of WN Virus.

In the first series of protection experiments we wished to establish whether immunization with pKUN1 DNA would protect mice against i.c. challenge with the wild type strain of KUN virus. Three weeks after a single immunization with pKUN1 or pKUN1 dGDD DNAs or with attenuated FLSD virus, mice were challenged i.c. with 1000 infectious units of the wild type KUN virus (MRM61C strain). Animals immunized with 1 or 10 ug of pKUN1 DNA or with 1000 i.u. of FLSD virus were solidly protected from challenge (80-100% survival), while unimmunized mice or those receiving defective pKUN1 dGDD DNA were highly susceptible to the challenge (only 20% survival) (Table 3). Survival of ⅖ (40%) of mice immunized with 0.1 ug of pKUN1 DNA indicated partial protection, but this result was not statistically significant.

To determine whether immunization with KUN DNA and attenuated FLSD virus would also protect mice from West Nile virus, immunized animals were challenged i.p. or i.c. with a lethal dose (20 i.u.) of the virulent New York strain (NY99 4132). Mice immunized with FLSD or 1 ug of pKUN 1 DNA were completely protected (100% survival) against i.c. challenge with West Nile virus, while 0.1 ug of pKUN1 DNA induced partial protection (Table 3.). On the other hand, unimmunized mice or those immunized with the defective KUN DNA were highly susceptible (90% mortality). Similarly, mice immunized with FLSD virus or pKUN 1 DNA were solidly protected from morbidity or mortality compared to controls when challenged i.p. with WN virus (Table 3).

The pKUN1 cDNA used according to the present invention is derived from a previously described FLSDX clone which differed from another closely related clone, pAKUN, by about 100,000 fold as measured by specific infectivity of corresponding in vitro transcribed RNA (15). We have previously confirmed the retention of the introduced mutation in NS2A gene (Arg175 to Lys) in the recovered AKUN virus (29), however the rest of the sequence of pAKUN cDNA and of the recovered AKUN virus, as well as of FLSDX cDNA (except for NS1 gene; 30) has not been determined. It was reasonable to assume that low specific infectivity of pAKUN RNA was due to the presence of mutations inhibiting virus replication and/or assembly, and that these mutations were corrected in the recovered AKUN virus. It was also likely that these inhibiting mutations originally present in pAKUN cDNA were corrected in FLSDX cDNA leading to dramatic improvement in specific infectivity of FLSDX RNA.

To confirm the above assumption we decided to determine the entire genomic sequence of pAKUN and FLSDX cDNA clones and compare them with the previously published sequence of MRM61C strain of KUN virus (Genebank accession No. D00246) (6). The differences between pAKUN, FLSDX and the published KUN sequence are shown in Table 4. FLSDX cDNA had 13 nucleotides different to the published KUN sequence with seven of them leading to amino acid changes; pAKUN cDNA had 17 nucleotides different to the published KUN sequence with 11 of them leading to amino acid changes. These amino acid mutations were scattered throughout entire coding region and were located in prM, E, NS1, NS2A, NS3 and NS4B genes. No mutations were found in NS2B, NS4A, NS5 and in the 5′ and 3′ UTRs. pAKUN and FLSDX cDNA clones had six common amino acid substitutions, mainly conserved, which were different from the original MRM61C published sequence (Table 4). These six common amino acids detected in both FLSDX and pAKUN sequences, aligned better with the sequences of other flaviviruses from the same subgroup than with the published MRM61C sequence, suggesting that they represent corrected errors in the originally published MRM61C sequence. The Pro250 to Leu codon change in the NS 1 gene was present in both pAKUN and FLSDX cDNAs and apparently contributed to delayed replication of recovered viruses (30). Thus, with the exclusion of one amino acid difference in NS1 (Pro250 to Leu) that apparently arose during cloning, FLSDX cDNA was identical to the corrected wt KUN RNA sequence.

One of two mutations in NS2A in pAKUN clone represented a conserved amino acid change (Arg175 to Lys) which was introduced intentionally as a marker mutation during plasmid construction. We demonstrated previously that this mutation was retained in the recovered AKUN virus and did not affect viral replication (29). Thus, it left only 3 amino acid codons in pAKUN cDNA different to those in FLSDX cDNAs and/or to those in corrected MRM61C viral RNA sequences (nucleotide 3701T to A, changing NS2A amino acid Ile59 to Asn; nucleotide 6163T to C, changing NS3 amino acid Tyr518 to His; nucleotide 6280T to C, changing NS3 amino acid Ser557 to Pro) (Table 4). These changes apparently contributed to the low specific infectivity of transcribed pAKUN RNA. To examine whether these mutations were present in RNA of the recovered AKUN virus we performed RT-PCR and sequence analysis of the corresponding genomic regions in viral RNA. The results showed that all these three mutations but not the marker mutation (Arg175 to Lys in NS2A) were reverted back to the wild type sequence in the recovered viral RNA (data not shown), clearly demonstrating the importance of these amino acid residues in virus replication.

### Example 2

### NS2A/A30P Mutant KUN Virus Displays Reduced Cytopathicity in Cells and Substantial (˜1000-Fold) Attenuation in Mice

Generation of NS2A/A30P KUN Virus.

NS2A/A30P virus was generated by introducing Ala30 to Pro mutation in NS2A into the full-length cDNA clone of KUN virus FLSDX(pro)HDVr (17). Mutant virus was produced in BHK cells after transfection with NS2A/A30P-mutated FLSDX(pro)HDVr RNA and titrated by plaque assay on Vero cells to determine the virus titre.

NS2A/A30P KUN Virus is Less Cytopathic in Monkey (Vero) Cells.

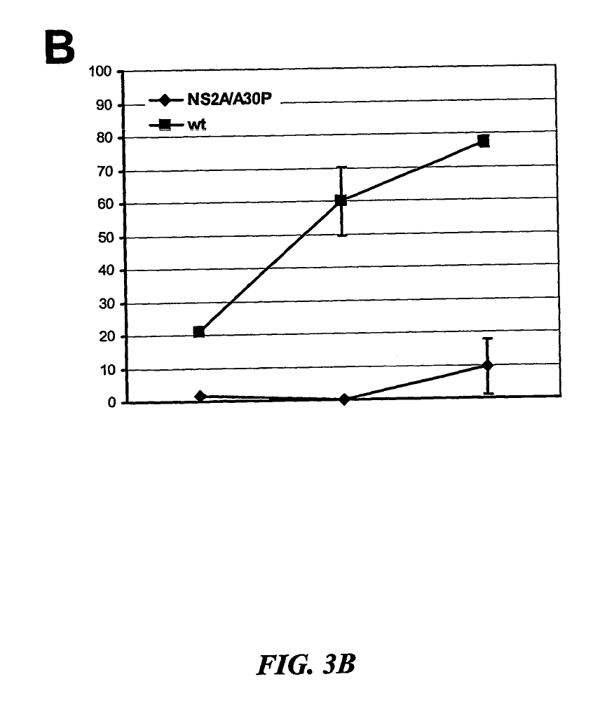

Infection of Vero cells with 0.01 MOI of NS2A/A30P virus produced no detectable cytopathic effect until 6 days post infection and only ˜10% cytopathicity by day 8 post infection (FIG. 3). In contrast, 20%, 60% and 80% cells were killed at days 4, 6, and 8 after infection with the same MOI of the wild type KUN virus (FIG. 3).

NS2A/A30P KUN Virus Induces Transcription of Substantially More Interferon-β mRNA in Human Cells than the Wild Type Virus.

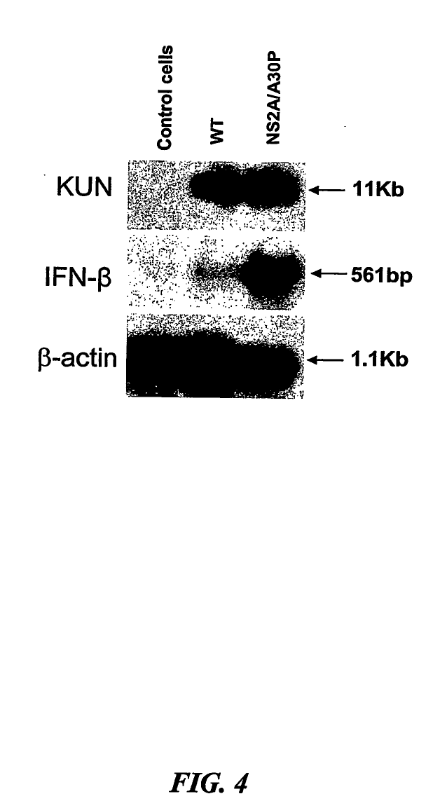

To access the effect of Ala30 to Pro mutation in NS2A on the induction of IFN-β mRNA transcription, human lung carcinoma cells A549 were infected with the mutant and the wild type virus at MOI=1 and examined for IFN-β mRNA at 24 h after infection. NS2A/A30P virus produced ˜6-fold more IFN-β mRNA than the wild type virus, while the amount of KUN RNA were similar (FIG. 4).

NS2A/A30P KUN Virus is at Least 1000-Fold Less Virulent than the Wild Type KUN Virus in Weanling Mice after Peripheral Inoculation

This virus represents a more attenuated KUNV mutant and produced no disease in highly susceptible 3 week old mice by i.p. inoculation at doses up to 10⁴ infectious units per mouse. In comparison, wild-type KUNV and the attenuated 250 Pro-Leu mutant (FLSD) produced disease in at least 50% of mice given=10 or=100 infectious units per dose respectively.

NS2A/A30P KUN Virus is at Least 1000-Fold Less Virulent than the Wild Type KUN Virus in Weanling Mice after Direct Inoculation into the Brain

Three week old mice are extremely susceptible to this route of inoculation, however the NS2A mutant exhibited a substantial reduction in virulence by this route. While 0.1-1.0 infectious units of wild type KUNV and 1.0-10 units of FLSD were required to produced disease by this route, 100-1000 units of the NS2A mutant were required to produce a similar level of disease.

Immunization of Mice with NS2A/A30P KUN Virus Induces Strong Humoral Immunity Comparable to that Induced by the FLSD Virus

Three week old mice inoculated i.p. with 10³ infectious units of the KUNV NS2A mutant produced strong antibody titres to KUNV as detected by ELISA (mean titre=320). Virus neutralisation titres to KUNV and WNV are yet to be determined, but based on previous data with KUNV FLSD, which produced similar ELISA titres, these responses are likely to neutralise and protect against NY99 WNV challenge.

### Example 3

### KUNV NS1 250 Ala Mutant Displays Substantial Attenuation in Mice and Reduced Growth Ability in Mosquito Cells

Virulence by Peripheral Inoculation

This virus represents a more attenuated KUNV mutant and produced no disease in highly susceptible 3 week old mice by i.p. inoculation at doses up to 10⁴ infectious units per mouse. In comparison, wild-type KUNV and the attenuated 250 Leu mutant (FLSD) produced disease in at least 50% of mice given=10 or=100 infectious units per dose respectively.

Virulence by Direct Inoculation into the Brain

Three week old mice are extremely susceptible to this route of inoculation, however the NS1 250 Ala mutant exhibited a substantial reduction in virulence by this route. While 0.1-1.0 infections units of wild type KUNV and 1.0-10 units of FLSD were required to produced disease by this route, 10-100 units of the NS1 250 Ala mutant were required to produce a similar level of disease.

Induction of Humoral Immunity in Mice by Peripheral Inoculation

Three week old mice inoculated i.p. with 10³ infectious units of the KUNV NS1 250 Ala mutant produced strong antibody titres to KUNV as detected by ELISA (mean titre =320). Sera from each mouse also neutralised KUNV. Neut titres to WNV NY99 are yet to be determined, but based on previous data with KUNV FLSD, which produced similar ELISA and Neut titres, these responses are likely to neutralise and protect against NY99 WNV.

Growth in Mosquito Cells

To determine whether mutant KUNV could infect and be transmitted by mosquitoes, we assessed their growth in a line of mosquito salivary gland cells. While wild type KUNV grew efficiently in these cells, FLSD, and to an even greater extent KUNV 250 Ala, grew very poorly, with infectious virus first detected in the cultures 6 and 12 hrs later respectively and reaching tires 10 and 100 fold lower respectively. The ability of the mutant viruses to infect whole mosquitoes is currently being evaluated.

### Discussion

Here we demonstrate that immunization of mice with plasmid DNA encoding the attenuated but infectious full-length KUN RNA provides protective immunity against challenge with the wild type KUN virus, and most importantly, against challenge with the highly pathogenic NY99 strain of West Nile virus. Apparently, replication of viral RNA produced in pKUN1 DNA-transfected cells and spread of the infectious progeny virus to nearby cells, allows sufficient virus amplification in the inoculated animal to produce a protective immune response that is similar in magnitude to that observed during natural virus infection. However, the limited replication of the attenuated pKUN1 progeny virus, and low amounts of infectious virus observed in mouse serum would likely preclude virus transmission to anthropod vectors, thus making the vaccine safer.

Our inability to detect virus in serum between 3 and 7 days post-immunization with the highest dose (10 ug) of pKUN1 DNA suggests that a rapid, transient viremia may have occurred during the first 72 h prior to the testing period. This is consistent with our detection of virus at only a single time point in serum after immunization with lug (day 3) and 0.1 ug doses (day 4). In contrast, i.p. injection of FLSD virus resulted in a viremia of at least 3 days duration (days 3-5). The difference between the routes of injection for plasmid DNA (i.m) and for virions (i.p.) may account for the observed differences in the longevity of viremia.

While as little as 0.1-1 ug of pKUN1 DNA was sufficient to induce protective immunity, mice immunized with 1-10 ug of the defective plasmid DNA construct (pKUN1dGDD) failed to generate a detectable humoral response. This suggests that the amounts of viral proteins produced from a messenger KUN RNA transcribed under direction of a CMV promoter are insufficient to induce protective immunity. This is consistent with previous reports using DNA-based subunit flavivirus vaccines expressing the prM-E structural genes of Japanese encephalitis virus (23), Murray Valley encephalitis virus (24) and WN virus (25). These studies reported that 50-100 ug doses were required to elicit protective immunity by a single injection via the i.m. route. The efficacy of the WN subunit vaccine could be significantly enhanced by performing co-electrotransfer on the inoculated muscle tissue immediately after the injection (25). This reduced the effective dose in mice to 1 or 0.1 ug, similar to the efficacy of the pKUN 1 DNA (injected into muscle without co-electrotransfer) observed in our study. It is also worth noting that a significant advantage of a vaccine expressing the full length flavivirus genome over the E-prM subunit, is that humoral responses to the NS1 protein and cell-mediated responses to NS3 epitopes may also contribute to more efficient viral clearance and protection (20, 26, 27).

In Australia, KUN virus is widespread and endemic in some areas. However, overt infection in man is extremely rare and nonfatal (10) demonstrating natural attenuation of this virus in humans.

Despite the degree of homology between KUN virus and WN virus, studies by Beasley et al. (11) have demonstrated that wild-type KUN virus is significantly less virulent in adult NIH Swiss mice than the New York strain of West Nile by peripheral inoculation. These findings agree with our inability to produce consistent mortality rates in adult BALB/c mice after inoculation with KUN virus by the i.p. route (results not shown). The inclusion of a stable proline to leucine (13) or alanine mutation at residue 250 in the NS1 protein, and/or a stable Alanine 30 to Proline substitution in the nonstructural protein NS2A, which attenuate KUN virus in mice, into the pKUN1 DNA vaccine backbone should result in further attenuation of the KUN virus-based vaccine, while retaining highly efficient protective immunity.

### Conclusions

The rapid spread of West Nile virus across the North American continent since 2002, has resulted in numerous fatal cases of human and equine disease (3, www.cdc.gov/ncidod/dvbid/westnile/index.htm). Consequently, an effective and safe vaccination strategy against the virus is urgently being sought for both medical and veterinary purposes. The naturally attenuated phenotype, stable genetics and well studied ecology, epidemiology and molecular biology of KUN virus provide a solid background for vaccine derivation. The safety of a KUN-based vaccine is further enhanced by the inclusion of a well characterized, stable attenuative mutation. The presented results demonstrating that mice vaccinated with pKUN1 DNA were protected from a challenge with the lethal doses of the New York strain of West Nile virus, suggest that immunization with KUN virus-based plasmid DNA may provide a rapid, convenient and effective vaccination strategy against further outbreaks of WN virus.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

### REFERENCES

1. Murgue, B. Zeller, H. and Deubel, V. (2002) Curr. Top. Microbiol. Immunol. 267, 195-221.
2. Lanciotti, R. S., Roehrig, J. T., Deubel, V., Smith, J., Parker, M., Steele, K., Volpe, K. E., Crabtree, J. H., Scherret, J. H., Hall, R. A., Mackenzie, J. S., Cropp, C. B., Panigrahy, B., Ostlund, E., Schmitt, B., Malkinson, M., Banet, C., Weisman, J., Komar, N., Savage, H. M., Stone, W., McNamara, T., & Gubler, D. J. (1999). Science 286, 2333-2337.
3. Roehrig, J. T., Layton, M., Smith, P., Campbell, G. L., Nasci, R. & Lanciotti, R. S. (2002). Curr. Top. Microbiol. Immunol. 267, 223-240.
4. Westaway, E. G. (1968). J. Immunol. 100, 569-580.
5. Hall, R. A., Burgess, G. W., Kay, B. H. and Clancy, P.(1991) Immunol. Cel. Biol. 69, 47-49.
6. Coia, G., Parker, M. D., Speight, G., Byrne, M. E. & Westaway, E. G. (1988). J. Gen. Virol. 69, 1-21.
7. Lanciotti, R. S., Ebel, G. D., Deubel, V., Kerst, A. J., Murri, S., Meyer, R., Bowen, M., McKinney, N., Morrill, W. E., Crabtree, M. B., Kramer, L. D., & Roehrig, J. T. (2002) Virology 298, 96-105.
8. Scherret, J. H., Poidinger, M., Mackenzie, J. S., Broom, A. K., Deubel, V., Lipkin, W. I., Briese, T., Gould, E. A., & Hall, R. A. (2001). Emerg. Infect. Dis. 7, 697-705.
9. Heinz, F. X., Collett, M. S., Purcell, R. H., Gould, E. A., Howard, C. R., Houghton, M., et al (2000) 7th Report of the International Committee for the Taxonomy of Viruses. Academic Press, San Diego, pp 859-878.
10. Hall, R. A., Broom, A. K., Smith, D. W., & Mackenzie, J. S. (2002). Curr. Top. Microbiol. Immunol. 267, 253-269.
11. Beasley, D. W., Li, L., Suderman, M. T., Barrett, A. D. (2002) Virology 296, 17-23.
12. Khromykh, A. A., Varnavski, A. N., Sedlak, P. L., Westaway, E. G. (2001) J. Virol. 75, 4633-4640.
13. Hall, R. A., Khromykh, A. A., Mackenzie, J. M., Scherret, J., Khromykh, T. I., Poidinger, M., & Mackenzie, J. S. (1999). Virology, 264, 66-75.
14. Ward, G., Rieder, E. & Mason P W. (1997) J. Virol. 71, 7442-7447.
15. Khromykh, A. A., Kenney, M. T. & Westaway, E. G. (1998). J. Virol. 72, 7270-7279.
16. Varnavski, A. N, Young, P. R., & Khromykh, A. A. (2000). J. Virol. 74, 4394-4403.
17. Liu W. J., Chen H. B, & Khromykh A. A. (2003) J. Virol. 77, 7804-7813.
18. Adams, S. C., Broom, A. K., Sammels, L. M., Hartnett, A. C., Howard, M. J., Coelen, R. J., Mackenzie, J. S. & Hall R. A. (1995). Virology 206, 49-56.
19. Hall, R. A., Broom, A. K., Howard, M. J., Hartnett, A. C. & Mackenzie, J. S. J. Virol. Meths. 51, 201-210.
20. Hall, R. A., Brand, T. N., Lobigs, M., Sangster, M. Y., Howard, M. J., Mackenzie, J. S. (1996) J. Gen. Virol. 77, 1287-1294.
21. Hall, R. A., Kay, B. H., Burgess, G. W., Clancy, P. & Fanning, I. D. (1990). J. Gen. Virol. 71, 2923-2930.
22. Anraku. I., Harvey, T. J., Linedale, R., Gardner, J., Harrich, D., Suhrbier, A., & Khromykh, A. A. (2002) J. Virol. 76, 3791-3799.
23. Chang, G. J., Hunt, A. R., & Davis, B. (2000) J. Virol. 74, 4244-4252.
24. Colombage, G., Hall, R., Pavy, M. & Lobigs, M. (1998) Virology, 250, 151-163.
25. Davis, B. S., Chang, G. J, Cropp, B., Roehrig, J. T., Martin, D. A., Mitchell, C. J., Bowen, R., & Bunning, M. L. (2001) J. Virol. 75, 4040-4047.
26. Parish, C. R., Coia, G., Hill, A., Mullbacher, A., Westaway, E. G. & Blanden, R. V. (1991) J. Gen. Virol., 72, 1645-1653.
27. Lobigs, M., Arthur, C. E., Mullbacher, A. & Blanden, R. V. (1993) Virology, 202, 195-201.
28. Westaway, E. G., Mackenzie, J. M., & Khromykh, A. A. (2002). Curr. Top. Microbiol. Immunol. 267, 324-351.
29. Khromykh, A. A., and E. G. Westaway. 1994. J. Virol. 68, 4580-4588.
30. Khromykh, A. A., A. N. Varnavski, and E. G. Westaway. 1998. J. Virol. 72, 5967-5977.
31 Lee, Hall and Lobigs, 2004, J. Virol, 78 8271-8280.

32. Liu, W. J., Chen, H. B., Wang, X. J., Huang, H., and A. A. Khromykh, 2004, J. Virol. 78 12225-12235.

TABLE 1
Isolation of KUN virus from mouse sera collected at various times
post injection with KUN DNAs or with attenuated FLSD virus

| | Days post immunisation^{#} | | | | |
|---|---|---|---|---|---|
| Immunogen | Day 3 | Day 4 | Day 5 | Day 6 | Day 7 |
| FLSD virus | +** | +** | +* | − | − |
| 0.1 ug pKUN1 | − | +** | − | − | − |
| 1 ug pKUN1 | +* | − | − | − | − |
| 10 ug pKUN1 | − | − | − | − | − |
| 10 ug pKUN1dGDD | − | − | − | − | − |
| Unimmunized | − | − | − | − | − |

^{#}Attenuated FLSD virus (1000 i.u.) was injected i.p., DNAs were injected i.m. Virus was detected in both (**) or one (*) of duplicate wells of C6/36 cells after secondary passage from cultures inoculated with pooled mouse serum.

TABLE 2
Detection of antibodies to KUN and WN viruses in sera* from
immunized mice by ELISA and by virus neutralization assays.

| | |
|---|---|
| Neutralization | |
| titers | ELISA titers |

| Mouse group | KUN | WN | KUN | WN |
|---|---|---|---|---|
| Unimmunized | <10 | <10 | <40 | <40 |
| 10 ug pKUN1dGDD | <10 | <10 | <40 | <40 |
| 0.1 ug pKUN1 | 10 | 10 | 160 | 80 |
| 1 ug pKUN1 | 20 | 20 | 320 | 80 |
| 10 ug pKUN1 | 20 | 20 | 320 | 80 |
| 1000 i.u. FLSD | 40 | 80 | 1280 | 640 |

*Mice were bled 19 days after a single immunization

TABLE 3
Protection of immunized mice from lethal challenge with NY99 WN and KUN viruses

| | % morbidity and mortality | | % mortality after i.c. | |
|---|---|---|---|---|
| | after i.p. challenge with | | challenge with KUN or | |
| | NY99 WN virus (20 i.u.)* | | NY99 WN viruses* | |
| | % morbidity** | % mortality^{@} | WN, 20 i.u. | KUN, 10³ i.u. |
| Mouse group | (sick/challenged) | (dead/challenged) | (dead/challenged) | (dead/challenged) |
| Control (unimmunized) | 100 (11/11) | 64 (7/11) | 89 (8/9) | 80 (4/5) |
| 10 ug pKUN1dGDD DNA# | ND | ND | ND | 80 (4/5) |
| 1 ug pKUN1dGDD DNA# | 75 (9/12) | 58 (7/12) | 90 (9/10) | ND |
| 0.1 ug pKUN1 DNA# | 16 (2/12) | 8 (1/12) | 33 (2/6) | 60 (3/5) |
| 1 ug pKUN1 DNA# | 8 (1/12) | 8 (1/12) | 0 (0/10) | 0 (0/5) |
| 10 ug pKUN1 DNA# | ND | ND | ND | 20 (1/5) |
| 1000 i.u. FLSD virus## | 0 (0/12) | 0 (0/12) | 0 (0/10) | 20 (1/5) |

*Mice were challenged i.p. or i.c. three weeks after a single immunization
**Percentage of mice that developed symptoms of fever (severely ruffled fur and lethargy)
^{@}Percentage of mice that progressed to symptoms of encephalitis (hunched posture, flaccid hindleg paralysis, moribund)
#Mice were immunized i.m. with varying doses of pKUN1 or pKUN1dGDD DNA at 4-5 weeks of age.
##Mice were immunized i.p. at 4-5 wks of age.
ND not don

TABLE 4
Differences between the published KUN MRM61C sequences and the sequences of infectious KUN cDNA clones

| | | Amino acid | | | | | | | |
|---|---|---|---|---|---|---|---|---|---|
| | | position in | MRM61C | | FLSDX cDNA | | pAKUN cDNA | | |
| Nucleotide | | encoded | Nucleo- | Amino | Nucleo- | Amino | Nucleo- | Amino | Corresponding reside in |
| position | Gene | protein | tide | acid | tide | acid | tide | acid | related flaviviruses |
| 544 | PrM | 28 | C^{a} | Pro | A | Thr | A | Thr | Thr (WN, JE, MVE) |
| 1500 | E | 178 | G | Leu | T^{c} | — | — | — | Leu(WN, JE)/Ala(MVE) |
| 1635 | E | 223 | C | Leu | — | — | T | — | Leu(WN, MVE, JE) |
| 1824 | E | 286 | C | Leu | G | — | G | — | Leu(WN, MVE, JE) |
| 2556 | NS1 | 29 | T^{a} | Ile | G | Met | G | Met | Ile(MVE)/Met (WN)/Val (JE) |
| 2924 | NS1 | 152 | A^{a} | Asn | G | Ser | G | Ser | Ser(WN, MVE, JE) |
| 3218 | NS1 | 250 | C | Pro | T^{b} | Leu | T^{b} | Leu | Pro(WN, MVE, JE) |
| 3701 | NS2A | 59 | T | Ile | — | — | A^{b} | Asn | Ile(WN, MVE)/Val (JE) |
| 4049 | NS2A | 175 | G | Arg | — | — | A^{c} | Lys | Arg(WN, MVE, JE) |
| 5937 | NS3 | 442 | A | Val | G | — | G | — | Val(WN, MVE, JE) |
| 5996 | NS3 | 462 | C^{a} | Thr | T | Ile | T | Ile | Ile (WN)/Val (JE, MVE) |
| 6017 | NS3 | 469 | C^{a} | Ala | T | Val | T | Val | Val (WN, JE)/Ile (MVE) |
| 6163 | NS3 | 518 | T | Tyr | — | — | C^{b} | His | Tyr (WN, MVE)/Phe (JE) |
| 6280 | NS3 | 557 | T | Ser | — | — | C^{b} | Pro | Ser (WN)/Gln (JE, MVE) |
| 7112 | NS4B | 66 | C^{a} | Thr | A | Asn | A | Asn | Asn (WN)/Thr(MVE, JE) |
| 7293 | NS4B | 126 | G | Val | A | — | A | — | Val(WN)/Leu(MVE, JE) |
| 7785 | NS5 | 35 | C | Val | T | — | T | — | Val(WN, MVE, JE) |
| 10197 | NS5 | 839 | C | Ser | T | — | T | — | Ser(WN)/Thr (JE, MVE) |

LEGEND TO TABLE 4
Nucleotides in bold in cDNA clones indicate changes from the original published sequence which lead to changes in corresponding amino acids, also shown in bold.
“—” shows no differences from wild type in the indicated nucleotide or in the corresponding encoded amino acid
^{a}apparent errors in the published MRM61C
^{b}mutations probably introduced during cloning
^{c}mutation introduced to produce cDNA clone
WN—west Nile virus, JE—Japanese encephalitis virus, MVE—Murray Valley encephalitis virus.
pAKUN and FLSDX cDNA sequences have been deposited to GeneBank and obtained accession numbers AY274505 and AY274504, respectively

1. An immunotherapeutic composition comprising an isolated nucleic acid capable of producing an infectious Kunjin virus together with a carrier, diluent or excipient, which upon administration to an animal, elicits a protective immune response to at least another flavivirus.

2. The immunotherapeutic composition of claim 1, wherein the isolated nucleic acid corresponds to substantially an entire genome of a Kunjin virus.

3. The immunotherapeutic composition of claim 2, wherein said isolated nucleic acid encodes at least one attenuating mutation.

4. The immunotherapeutic composition of claim 2, wherein said isolated nucleic acid encodes at least one attenuating mutation in a Kunjin virus non-structural protein.

5. The immunotherapeutic composition of claim 4, wherein the attenuating mutation is located at an amino acid residue selected from the group consisting of:
Proline residue 250 of nonstructural protein NS 1; Alanine residue 30 of nonstructural protein NS2A; Asparagine residue 101 of nonstructural protein NS2A; and Proline residue 270 of nonstructural protein NS5.

6. The immunotherapeutic composition of claim 5, wherein the mutation is selected from the group consisting of: Proline residue 250 of the NS1 protein substituted by an amino acid selected from the group consisting of leucine, valine and alanine; Alanine 30 substituted by Proline in the nonstructural protein NS2A; Asparagine 101 substituted by Aspartate in the nonstructural protein NS2A; and Proline 270 substituted by Serine in the nonstructural protein NS5.

7. The immunotherapeutic composition of claim 2, wherein said isolated nucleic acid encodes at least one attenuating mutation in a Kunjin virus structural protein.

8. The immunotherapeutic composition of claim 7, wherein the Kunjin virus structural protein is E protein.

9. The immunotherapeutic composition of claim 8, wherein the attenuating mutation is located an amino acid residue selected from the group consisting of: residue 49, residue 138, residue 306 and residue 390 of E protein.

10. The immunotherapeutic composition of claim 9, wherein the attenuating mutation is Glu390 to Gly.

11. The immunotherapeutic composition of claim 1, wherein the isolated nucleic acid is DNA that is operably linked to a promoter so that the DNA is expressible in a mammalian cell.

12. The immunotherapeutic composition of claim 1, wherein the isolated nucleic acid is RNA.

13. The immunotherapeutic composition of claim 12, wherein the RNA is packaged into virions

14. The immunotherapeutic composition of claim 1, wherein said at least another flavivirus is more pathogenic than Kunjin virus.

15. The immunotherapeutic composition of claim 14, wherein said at least anther flavivirus is a West Nile virus.

16. The immunotherapeutic composition of claim 15, wherein said West Nile virus is NY99 strain West Nile virus.

17. A method of immunizing an animal including the step of administering an isolated nucleic acid capable of producing an infectious Kunjin virus to said animal to thereby elicit a protective immune response to at least another flavivirus.

18. The method of claim 17, wherein the isolated nucleic acid corresponds to substantially an entire genome of a Kunjin virus.

19. The method of claim 18, wherein said isolated nucleic acid encodes at least one attenuating mutation.

20. The method of claim 19, wherein the said isolated nucleic acid is DNA operably linked to a promoter operable in a mammalian cell.

21. The method of claim 19, wherein the isolated nucleic acid is RNA.

22. The method of claim 21, wherein the RNA is packaged in virions.

23. The method of claim 17, wherein said at least another flavivirus is more pathogenic than Kunjin virus.

24. The method of claim 23, wherein said at least another flavivirus is a West Nile virus.

25. The method of claim 24, wherein said West Nile virus is NY99 strain West Nile virus.

26. The method of claim 17, wherein the animal is a mammal.

27. The method of claim 26, wherein the mammal is an equine.

28. The method of claim 26, wherein the mammal is a human.

29. The method of claim 17, wherein the animal is an avian.

30. A non-human animal immunized according to the method of claim 17.

31. The non-human animal of claim 30, which is an equine.

32. The non-human animal of claim 30, which is an avian.

33. An immunocompetent biological material isolated from an animal immunized according to claim 17.

34. The immunocompetent biological material of claim 33, which comprises one or more lymphocytes and/or antigen-presenting cells.

35. The immunocompetent biological material of claim 34, which comprises plasma and/or serum.

36. A method of using Kunjin virus to identify another flavivirus against which Kunjin virus is suitable for use as an immunogen, said method including the steps of:
(i) administering an isolated nucleic acid capable of producing an infectious Kunjin virus to an animal; and
(ii) determining whether said animal is protectively immunized against infection by another flavivirus;
wherein if said animal is protectively immunized against said another flavivirus, Kunjin virus is suitable for use as an immunogen against said another flavivirus.

37. The method of claim 36, wherein said another flavivirus is more pathogenic than Kunjin virus.
